(19) United States
(12) Patent Application Publication
(10) Pub. No.: US 2006/0025334 A1
(43) Pub. Date: Feb. 2, 2006

(54) Vanilloid receptor-2 ligands for treating anxiety or depression

(76) Inventors: Kevin Russel Oliver, Huntingdon (GB); Guy Ralph Seabrook, Blue Bell, PA (US); Anna Wainwright, Maclesfield (GB)

Correspondence Address: MERCK AND CO., INC, P O BOX 2000, RAHWAY, NJ, 07065-0907, US

(21) Appl. No.: 10/535,091

(22) PCT Filed: Nov. 18, 2003
(86) PCT No.: PCT/GB03/04988

§ 371 Date: May 13, 2005

(30) Foreign Application Priority Data
Nov. 18, 2002 (GB) 0226850.6
Nov. 18, 2002 (GB) 0226865.4
Oct. 1, 2003 (GB) 0322990.3

(51) Int. Cl. A61K 38/17; A61K 48/00; A61K 31/16

(52) U.S. Cl. 514/12; 514/44; 514/630

(57) ABSTRACT

The present invention relates to the use of a compound selected from: (a) a VR2 polypeptide; (b) a compound which modulates the activity of a VR2 polypeptide; (c) a polynucleotide encoding a VR2 polypeptide; or (d) an antisense polynucleotide to a polynucleotide encoding a VR2 polypeptide, for the manufacture of a medicament for treating anxiety and/or depression, circadian rhythm disorders, pre-term labor, erectile dysfunction, hypertension and/or schizophrenia.

This invention relates to new uses for polynucleotides and polypeptides encoded by them, to their use in therapy and to agonists, antagonists and/or inhibitors thereof which are useful in therapy.

In particular, the present invention relates to the vanilloid 2 receptor (hereinafter VR2) polypeptide, otherwise known as TRPV2, VRL, VRL-1, Vanilrep2, VRCC, VRRP-1, GRC or SAC2b.

Several patent applications describing cDNA encoding human VR2 have been published: WO 99/37675 (published 29 Jul. 1999), WO 99/37765 (published 29 Jul. 1999), WO 99/46377 (published 16 Sep. 1999), WO 00/22121 (published 20 Apr. 2000), GB-2,346,882 (published 23 Aug. 2000), WO 01/34805 (published 17 May 2001), WO 01/46258 (published 28 Jun. 2001) and EP-1 160 254 (published 12 Dec. 2001).

More particularly, the present invention relates to new uses of the VR2 polypeptide, to new uses for compounds which modulate the activity of a VR2 polypeptide, to new uses of a polynucleotide encoding a VR2 polypeptide, and to new uses of antisense polynucleotides to a polynucleotide encoding a VR2 polypeptide. Such uses include the treatment of anxiety and/or depression and associated disorders and/or circadian rhythm disorders. In a further aspect, the invention relates to methods for treating conditions associated with VR2 imbalance or mutation, with the compounds which modulate the activity of a VR2 polypeptide, for example, as agonists, antagonists and/or inhibitors thereof.

Anxiety is an emotional condition characterised by feelings such as apprehension and fear accompanied by physical symptoms such as tachycardia, increased respiration, sweating and tremor. It is a normal emotion but when it is severe and disabling it becomes pathological.

Depression is generally characterised by the presence of major depressive episodes which are defined as being a period of at least two weeks during which, for most of the day and nearly every day, there is either depressed mood or the loss of interest or pleasure in all, or nearly all activities. The individual may also experience changes in appetite or weight, sleep and psychomotor activity; decreased energy; feelings of worthlessness or guilt; difficulty thinking, concentrating or making decisions; and recurrent thoughts of death or suicidal ideation, plans or attempts. One or more major depressive episodes may give rise to a diagnosis of major depressive disorder (Diagnostic and Statistical Manual of Mental Disorders, Fourth Edition, American Psychiatric Association, 1994).

Circadian rhythm disorders are described in detail, for example, in International Patent Specification No. WO 98/02158. Such disorders are responsible for a variety of clinical conditions including time-zone change (jet-lag) syndrome, shift-work sleep disorder, delayed sleep-phase syndrome, advanced sleep-phase syndrome, and non-24-hour sleep-wake disorder. Disturbances of sleep (or sleep disorders) are a particular example of a circadian rhythm disorder that affect a subject's ability to fall and/or stay asleep, and involve sleeping too little, too much or result in abnormal behaviour associated with sleep.

Further new uses include the treatment of disorders associated with the function (or misfunction) of vasopressin and/or oxytocin, including water retention, lactatory activity and modulation of uterine contraction in the female, and erectile function in the male. A further use includes the treatment of schizophrenia. In a further aspect, the invention relates to methods for treating conditions associated with VR2 imbalance or mutation, with the compounds which modulate the activity of a VR2 polypeptide, for example, as agonists, antagonists and/or inhibitors thereof.

Most especially, the present invention relates to the treatment or prevention of pre-term labour, erectile dysfunction and/or hypertension, and associated disorders, as well as schizophrenia. Even more especially, the present invention relates to the treatment or prevention of pre-term labour, erectile dysfunction and/or hypertension and associated disorders.

The present invention is based on the surprising finding that VR2 is expressed at significantly higher levels in certain regions of the CNS than in a wide range of other regions and tissues tested. In particular, localisation is in the hypothalamus, and predominantly in the paraventricular and supraoptic nuclei.

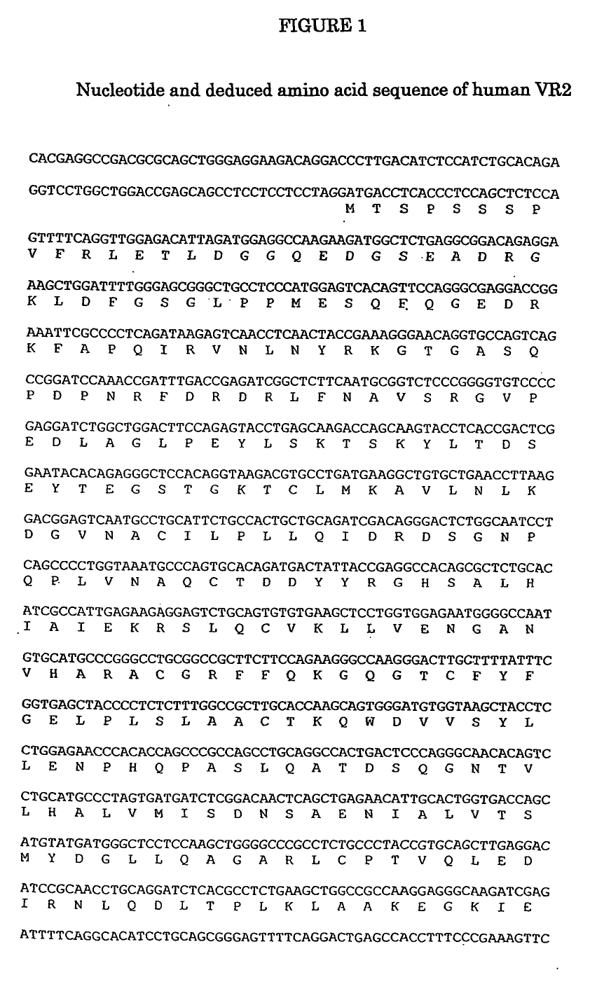

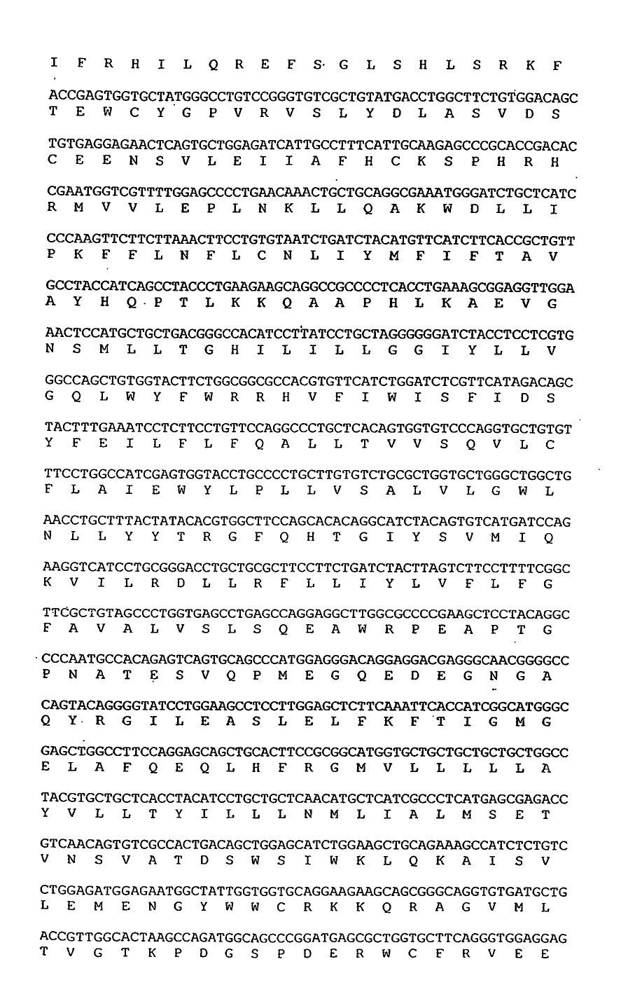

FIG. 1 shows the nucleic acid sequence (coding region of SEQ ID NO: 1) and the predicted amino acid sequence (SEQ ID NO: 2) for VR2.

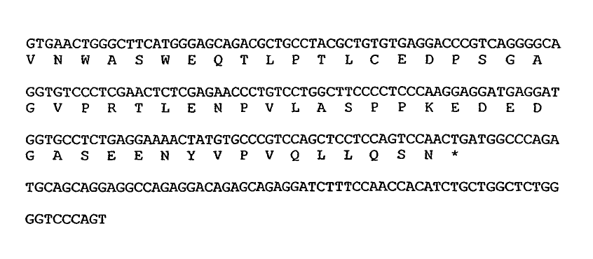

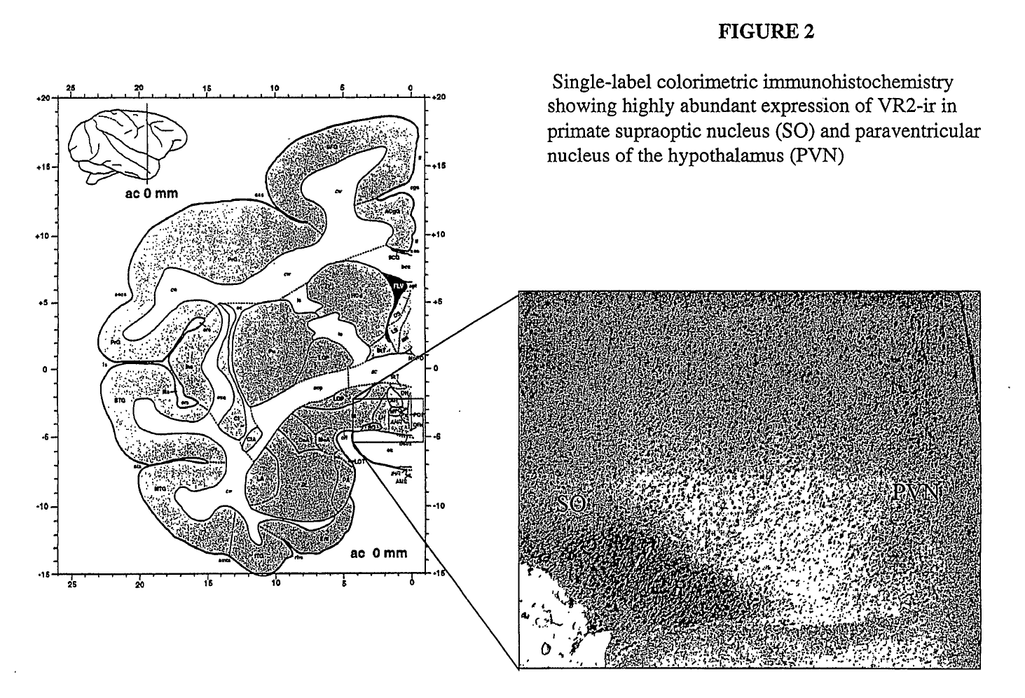

FIG. 2 shows the results of single-label colorimetric immunohistochemistry showing localisation of VR2 immunoreactivity in primate supraoptic nucleus (SON) and paraventricular nucleus of the hypothalamus (PVN).

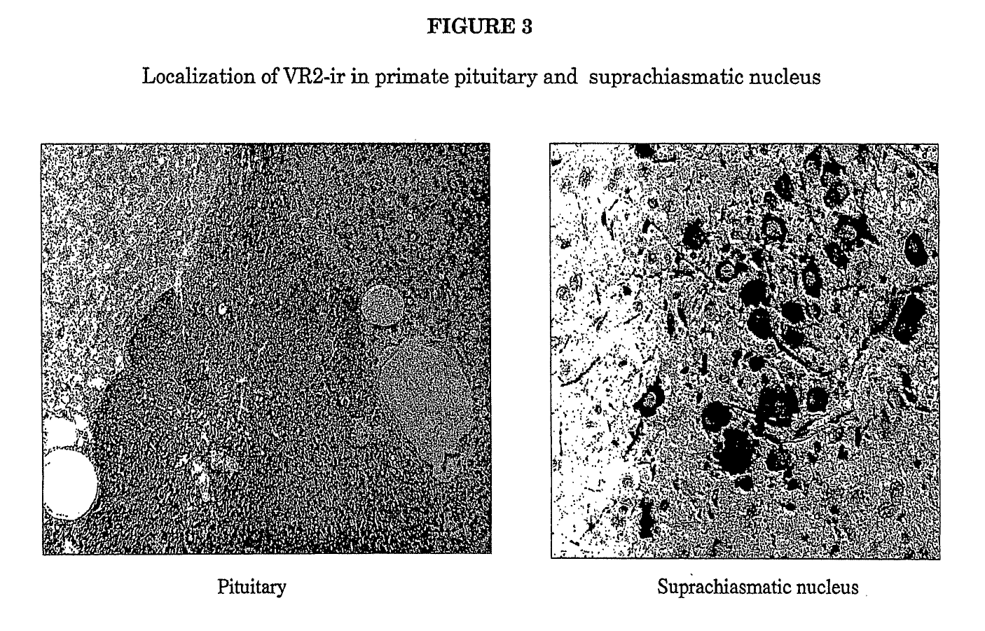

FIG. 3 shows the results of single-label colorimetric immunohistochemistry showing localisation of VR2 immunoreactivity in primate pituitary and suprachiasmatic nucleus.

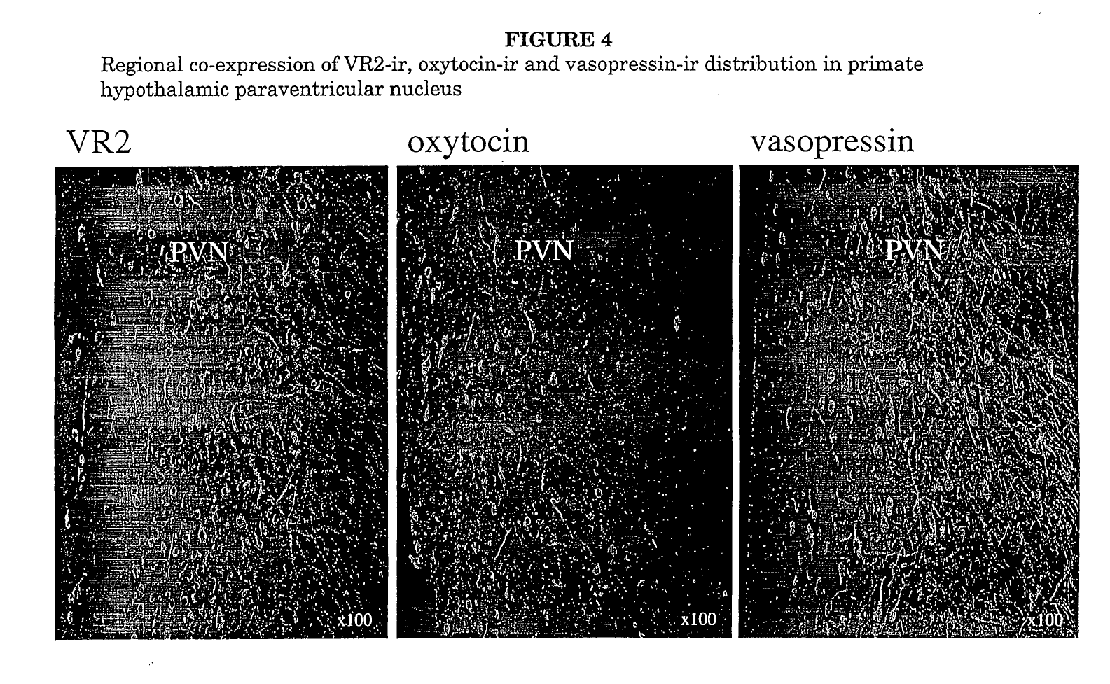

FIG. 4 shows the results of immunofluorescence microscopy showing regional co-expression of VR2, oxytocin and vasopressin in primate hypothalamic paraventricular nucleus.

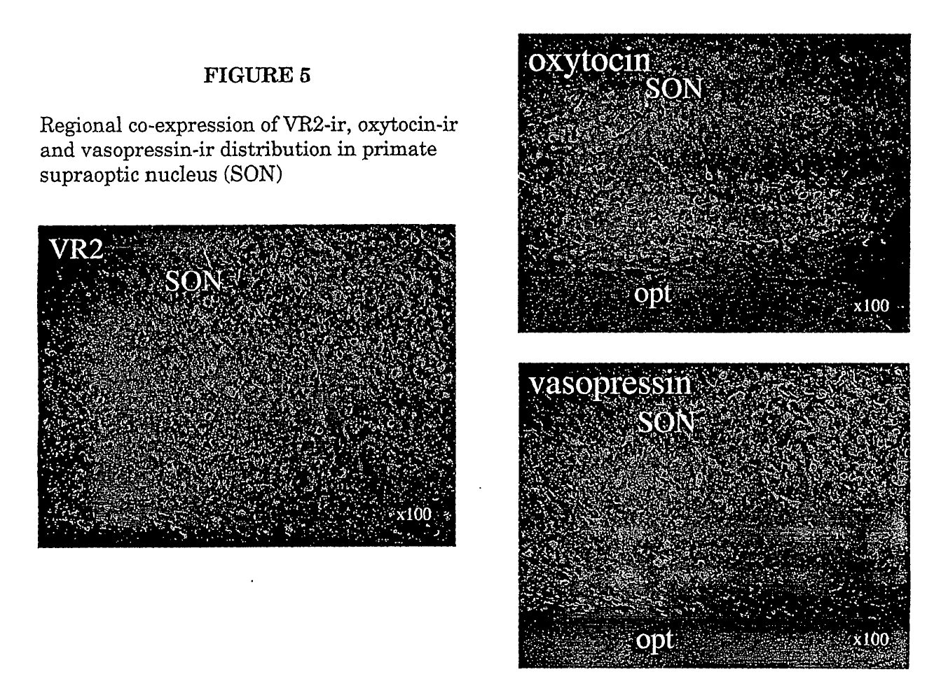

FIG. 5 shows the results of immunofluorescence microscopy showing regional co-expression of VR2, oxytocin and vasopressin in primate supraoptic nucleus (SON).

Thus in a first aspect, the present invention relates to the use of a compound selected from:
(a) a VR2 polypeptide;
(b) a compound which modulates the activity of a VR2 polypeptide;
(c) a polynucleotide encoding a VR2 polypeptide; or
(d) an antisense polynucleotide to a polynucleotide encoding a VR2 polypeptide,
   for the manufacture of a medicament for treating anxiety and/or depression and/or circadian rhythm disorders.

In an alternative aspect of the present invention, there is provided a method for the treatment of anxiety and/or depression and/or circadian rhythm disorders which comprises administration of an effective amount of a compound selected from:
(a) a VR2 polypeptide;
(b) a compound which modulates the activity of a VR2 polypeptide;
(c) a polynucleotide encoding a VR2 polypeptide; or
(d) an antisense polynucleotide to a polynucleotide encoding a VR2 polypeptide,
   to a patient in need of such treatment.

Thus in a second aspect, the present invention relates to the use of a compound selected from:
(a) a VR2 polypeptide;
(b) a compound which modulates the activity of a VR2 polypeptide;
(c) a polynucleotide encoding a VR2 polypeptide; or
(d) an antisense polynucleotide to a polynucleotide encoding a VR2 polypeptide,
   for the manufacture of a medicament for treating pre-term labour, erectile dysfunction and/or hypertension, and associated disorders, or schizophrenia. Particularly the use is for treating pre-term labour, erectile dysfunction and/or hypertension, and associated disorders.

In an alternative aspect of the present invention, there is provided a method for the treatment of pre-term labour, erectile dysfunction and/or hypertension, and associated disorders or schizophrenia which comprises administration of an effective amount of a compound selected from:
(a) a VR2 polypeptide;
(b) a compound which modulates the activity of a VR2 polypeptide;
(c) a polynucleotide encoding a VR2 polypeptide; or
(d) an antisense polynucleotide to a polynucleotide encoding a VR2 polypeptide,
   to a patient in need of such treatment. In particular the method is for the treatment of pre-term labour, erectile dysfunction and/or hypertension, and associated disorders.

Compounds which modulate the activity of a VR2 polypeptide include compounds that activate the VR2 polypeptide and also compounds which inhibit the activity of a VR2 polypeptide. Compounds which inhibit the activity of a VR2 polypeptide are particularly preferred.

VR2 polypeptides for use in the invention include isolated polypeptides comprising an amino acid sequence which has at least 95% identity, preferably at least 97 to 99% identity, to that of SEQ ID NO: 2. Such polypeptides include those comprising the amino acid of SEQ ID NO: 2.

Further, VR2 polypeptides include isolated polypeptides in which the amino acid sequence has at least 95% identity, preferably at least 97 to 99% identity, to the amino acid sequence of SEQ ID NO: 2. Such polypeptides include the polypeptides of SEQ ID NO: 2.

Still further, VR2 polypeptides include isolated polypeptides encoded by a polynucleotide comprising the sequence contained in SEQ ID NO: 1.

The VR2 polypeptides may be in the form of the “mature” protein or may be a part of a larger protein such as a precursor or a fusion protein. It is often advantageous to include an additional amino acid sequence which contains secretory or leader sequences, pro-sequences, sequences which aid in purification such as multiple histidine residues, or an additional sequence for stability during recombinant production.

The VR2 polypeptides can be prepared in any suitable manner. Such polypeptides include isolated naturally occurring polypeptides, recombinantly produced polypeptides, synthetically produced polypeptides, or polypeptides produced by a combination of these methods. Means for preparing such polypeptides are well understood in the art.

For preparing VR2 polypeptides by recombinant means, a polynucleotide encoding a VR2 polypeptide can be used (hereinafter a “VR2 polynucleotide”).

VR2 polynucleotides may be obtained, using standard cloning and screening techniques (Sambrook et al., Molecular Cloning: A Laboratory Manual, 2nd Ed., Cold Spring Harbor Laboratory Press, Cold Spring Harbor, N. Y.(1989) and United Kingdom patent publication No. 2,346,882 in the name of Merck Sharp & Dohme Limited) from a cDNA library derived from mRNA in cells of human brain. VR2 polynucleotides can also be obtained from natural sources such as genomic DNA libraries or can be synthesised using well-known and commercially available techniques.

GB-A-2,346,882 further discloses methods for the recombinant production of VR2 polypeptides, including expression vectors and hosts and details of purification methods.

VR2 polypeptides or their fragments or analogs thereof, or cells expressing them, can also be used as immunogens to produce antibodies immunospecific for polypeptides of the present invention. The term “immunospecific” means that the antibodies have substantially greater affinity for the polypeptides of the invention than their affinity for other related polypeptides in the prior art.

Antibodies generated against VR2 polypeptides may be obtained by administering the polypeptides or epitope-bearing fragments, analogs or cells to an animal, preferably a non-human animal, using routine protocols. For preparation of monoclonal antibodies, any technique which provides antibodies produced by continuous cell line cultures can be used.

Examples include the hybridoma technique (Kohler, G. and Milstein, C., Nature (1975) 256:495-497), the trioma technique, the human B-cell hybridoma technique (Kozbor et al., Immunology Today (1983) 4:72) and the EBV-hybridoma technique (Cole et al., Monoclonal Antibodies and Cancer Therapy, 77-96, Alan R. Liss. Inc., 1985).

Techniques for the production of single chain antibodies, such as those described in U.S. Pat. No. 4,946,778, can also be adapted to produce single chain antibodies to polypeptides of this invention. Also, transgenic mice, or other organisms, including other mammals, may be used to express humanised antibodies.

Antibodies against polypeptides of the present invention may be employed to treat anxiety and/or depression in accordance with the present invention.

Antibodies against polypeptides of the present invention may be employed to treat pre-term labour, erectile dysfunction and/or hypertension, and associated disorders or schizophrenia in accordance with the present invention. Antibodies may particularly be employed to treat pre-term labour, erectile dysfunction and/or hypertension, and associated disorders.

VR2 polypeptides can be used to devise screening methods to identify compounds which modulate the activity of said VR2 polypeptides. Such modulators include compounds which stimulate (agonists) or inhibit (antagonists) the function of the VR2 polypeptides. In general, modulators of VR2, such as agonists or antagonists, may be employed for therapeutic and prophylactic purposes for pre-term labour, erectile dysfunction and/or hypertension, and associated disorders or schizophrenia, and particularly for pre-term labour, erectile dysfunction and/or hypertension, and associated disorders. In general, modulators of VR2, such as agonists or antagonists, may be employed for therapeutic and prophylactic purposes for anxiety and/or depression. Antagonists (or inhibitors) of VR2 are particularly preferred. Compounds may be identified from a variety of sources, for example, cells, cell-free preparations, chemical libraries, and natural product mixtures. Such modulators so-identified may be natural or modified substrates, ligands or receptors of the VR2 polypeptides; or may be structural or functional mimetics thereof (see Coligan et al., Current Protocols in Immunology 1(2): Chapter 5 (1991)).

The screening method may simply measure the binding of a candidate compound to the VR2 polypeptides, or to cells or membranes bearing the VR2 polypeptide, or a fusion protein thereof by means of a label directly or indirectly associated with the candidate compound. Alternatively, the screening method may involve competition with a labelled competitor. Further, these screening methods may test whether the candidate compound results in a signal generated by activation or inhibition of the VR2 polypeptides, using detection systems appropriate to the cells bearing the VR2 polypeptide. Inhibitors of activation are generally assayed in the presence of a VR2 agonist, and the effect on activation by the agonist by the presence of the candidate compound is observed. Constitutively active polypeptides may be employed in screening methods for inverse agonists or inhibitors, in the absence of an agonist or inhibitor, by testing whether the candidate compound results in inhibition of activation of the VR2 polypeptide. Further, the screening methods may simply comprise the steps of mixing a candidate compound with a solution containing a VR2 polypeptide to form a mixture, measuring VR2 activity in the mixture, and comparing the VR2 activity of the mixture to a standard. Fusion proteins, such as those made from Fc portion and VR2 polypeptide, as hereinbefore described, can also be used for high-throughput screening assays to identify antagonists for the polypeptide of the present invention (see D. Bennett et al., J. Mol. Recognition, 8:52-58 (1995); and K. Johanson et al., J. Biol. Chem., 270(16):9459-9471 (1995)).

The polynucleotides, polypeptides and antibodies to the VR2 polypeptides may also be used to configure screening methods for detecting the effect of added compounds on the production of MRNA and polypeptide in cells. For example, an ELISA assay may be constructed for measuring secreted or cell associated levels of polypeptide using monoclonal and polyclonal antibodies by standard methods known in the art. This can be used to discover agents which may inhibit or enhance the production of polypeptide (also called antagonist or agonist, respectively) from suitably manipulated cells or tissues.

Examples of potential polypeptide antagonists include antibodies or, in some cases, oligonucleotides or proteins which are closely related to the ligands, substrates or receptors of the VR2 polypeptide, e g, a fragment of the ligands, substrates or receptors or small molecules which bind to the VR2 polypeptides of the present invention but do not elicit a response, so that the activity of the VR2 polypeptide is prevented.

It will be readily appreciated by the skilled artisan that a VR2 polypeptide may also be used in a method for the structure-based design of a compound that modulates the activity of the VR2 polypeptide by:
(a) determining in the first instance the three-dimensional structure of the VR2 polypeptide;
(b) deducing the three-dimensional structure for the likely reactive or binding site(s) of a modulating compound;
(c) synthesising candidate modulating compounds that are predicted to bind to or react with the deduced binding or reactive site; and
(d) testing whether the candidate compounds are indeed modulators.

It will be further appreciated that this will normally be an iterative process.

In a further aspect, the present invention provides methods for treating anxiety, and in particular anxiety disorders including, but not limited to, panic disorder with or without agoraphobia, agoraphobia without history of panic disorder, specific phobias, social phobias and obsessive-compulsive disorder.

“Panic disorder” is defined as the presence of recurrent panic attacks followed by at least one month of persistent concern about having another panic attack. A “panic attack” is a discrete period in which there is a sudden onset of intense apprehension, fearfulness or terror. During a panic attack, the individual may experience a variety of symptoms including palpitations, sweating, trembling, shortness of breath, chest pain, nausea and dizziness. Panic disorder may occur with or without agoraphobia.

“Phobias” includes agoraphobia, specific phobias and social phobias. “Agoraphobia” is characterised by an anxiety about being in places or situations from which escape might be difficult or embarrassing or in which help may not be available in the event of a panic attack. Agoraphobia may occur without history of a panic attack. A “specific phobia” is characterised by clinically significant anxiety provoked by exposure to a specific feared object or situation. Specific phobias include the following subtypes: animal type, cued by animals or insects; natural environment type, cued by objects in the natural environment, for example storms, heights or water; blood-injection-injury type, cued by the sight of blood or an injury or by seeing or receiving an injection or other invasive medical procedure; situational type, cued by a specific situation such as public transportation, tunnels, bridges, elevators, flying, driving or enclosed spaces; and other type where fear is cued by other stimuli. Specific phobias may also be referred to as simple phobias. A “social phobia” is characterised by clinically significant anxiety provoked by exposure to certain types of social or performance circumstances. Social phobia may also be referred to as social anxiety disorder.

“Obsessive-compulsive disorder is characterised by recurrent obsessions or compulsions that are severe enough to be time consuming (i.e. they take at least one hour a day) or cause marked distress or significant impairment. At some point during the course of the disorder, the patient should recognise that the obsessions or compulsions are excessive or unreasonable.

Other anxiety disorders encompassed within the term “anxiety disorders” include anxiety disorders induced by alcohol, amphetamines, caffeine, cannabis, cocaine, hallucinogens, inhalants, phencyclidine, sedatives, hypnotics, anxiolytics and other substances, and adjustment disorders with anxiety.

In a further aspect, the present invention provides methods for treating mood disorders, and in particular depression, especially major depressive disorders includes single or recurrent major depressive episodes, with or without psychotic features, catatonic features, melancholic features, atypical features or postpartum onset and, in the case of recurrent episodes, with or without interepisode recovery and with or without seasonal pattern.

Other mood disorders encompassed within the term “major depressive disorder” include dysthymic disorder with early or late onset and with or without atypical features; dementia of the Alzheimer's type, with early or late onset, with depressed mood; vascular dementia with depressed mood; mood disorders induced by alcohol, amphetamines, cocaine, hallucinogens, inhalants, opioids, phencyclidine, sedatives, hypnotics, anxiolytics and other substances; schizoaffective disorder of the depressed type; and adjustment disorder with depressed mood.

Major depressive disorders may also result from a general medical condition including, but not limited to, myocardial infarction, diabetes, miscarriage or abortion, etc.

A “major depressive episode” is defined as at least two weeks of depressed mood or loss of interest, which may be accompanied by other symptoms of depression. The symptoms must persist for most of the day (i.e. for at least two thirds of the patients' waking hours), nearly every day (i.e. for at least ten out of fourteen days) for at least two consecutive weeks. A “depressed mood” is often described by the patient as feeling sad, hopeless, helpless or worthless. The patient may also appear sad to an observer, for example, through facial expression, posture, voice and tearfulness. In children and adolescents, the mood may be irritable. A “loss of interest” is often described by the patient as feeling less interested in hobbies or not feeling any enjoyment in activities that were previously considered to be pleasurable.

A major depressive episode may be accompanied by other symptoms of depression including significant weight loss when not dieting or weight gain (e.g. a change of more than 5% body weight in one month), or decrease or increase in appetite; insomnia or hypersomnia; psychomotor agitation or retardation; fatigue or loss of energy; feelings of worthlessness or excessive or inappropriate guilt; diminished ability to think or concentrate; or indecisiveness; and recurrent thoughts of death, recurrent suicidal ideation with or without a specific plan, or a suicide attempt.

In a further aspect, the present invention provides methods for achieving a chronobiologic (circadian rhythm phase-shifting) effect and thereby alleviating circadian rhythm disorders. The present invention is further directed to methods for blocking the phase-shifting effects of light in a mammal.

In particular, the present invention provides a method for the phase advance or phase delay in the circadian rhythm of a subject.

The present invention is further directed to methods for enhancing or improving sleep quality as well as preventing and treating sleep disorders and sleep disturbances in a mammal. In particular, the present invention provides a method for enhancing or improving sleep quality by increasing sleep efficiency and augmenting sleep maintenance. In addition, the present invention provides a method for preventing and treating sleep disorders and sleep disturbances in a mammal. The present invention is useful for the treatment of sleep disorders, including Disorders of Initiating and Maintaining Sleep (insomnias) (“DIMS”) which can arise from psychophysiological causes, as a consequence of psychiatric disorders (particularly related to anxiety), from drugs and alcohol use and abuse (particularly during withdrawal stages), childhood onset DIMS, nocturnal myoclonus and restless legs and non specific REM disturbances as seen in ageing.

Pre-term labour is a major obstetric problem because of the high incidence of neonatal mortality or long-term handicap associated with it. Approximately 6-10% of all births in the UK are premature in nature and pre-term delivery is responsible for 70-85% of neonatal morbidity and mortality. Currently available drugs are poorly effective and have potentially serious side effects, for the mother and/or foetus. In a further aspect of the present invention, there is therefore provided methods of inducing myometrial relaxation, preventing pre-term labour, stopping labour, for example, in preparation for a Caesarean delivery, and/or treating or preventing dysmenorrhea.

Impotence can be defined literally as a lack of power, in the male, to copulate and may involve an inability to achieve penile erection or ejaculation, or both. More specifically, erectile impotence or dysfunction may be defined as an inability to obtain or sustain an erection adequate for intercourse. Its prevalence is claimed to be between 2 and 7% of the human male population, increasing with age, up to 50 years, and between 18 and 75% between 55 and 80 years of age. In the USA alone, for example, it has been estimated that there are up to 10 million impotent males, with the majority suffering from problems of organic rather than of psychogenic origin. One third of older men receiving medical treatment also has difficulty with erectile function. Risk factors for this disorder include atherosclerosis, hypertension, diabetes mellitus, depression and other psychiatric disorders, hypogonadism, pelvic surgery, kidney failure, multiple sclerosis, stroke, epilepsy, and alcoholism. In a further aspect of the present invention, there is therefore provided methods for the treatment or prevention of erectile dysfunction and/or impotence.

Twenty percent of the adult population suffers from chronic hypertension and 70% of patients with type 2 diabetes are hypertensive. High blood pressure is an important component in the development of atherosclerosis and other illnesses. In a further aspect of the present invention, there is therefore provided methods for the treatment or prevention of hypertension and/or congestive heart failure, inducing diuresis, and/or inhibiting platelet agglutination.

Blood pressure problems (e.g. hypertension and hypertension induced pre-eclampsia) are one of the most common reasons women are admitted to hospital during pregnancy, particularly during the final trimester. If untreated, pre-eclampsia can lead to eclampsia, which is very dangerous. Pre-eclampsia and eclampsia are the most important causes of death during pregnancy in the UK, USA and Nordic countries. Between 5 and 10% of women in their first pregnancies develop pre-eclampsia. There are also corresponding risks for the foetus. Few, if any, treatments are currently available for this condition and often the pregnant mother is forced to be hospitalised. Early induction of labour is often seen as a necessity in mothers suffering from eclampsia. In a further aspect of the present invention, there is therefore provided methods for the treatment or prevention of pre-eclampsia and/or eclampsia.

Where appropriate, however, it will also be appreciated that a VR2 agonist may be of use in the induction of labour if necessary.

As used herein the term “mammal” includes animals of economic importance such as bovine, ovine, and porcine animals, especially those that produce meat, as well as domestic animals, sports animals, zoo animals, and humans, the latter being preferred.

As used herein, the term “treatment” refers both to the treatment and, unless otherwise stated, to prevention or prophylactic therapy to prevent occurrence or recurrence of the aforementioned conditions.

If the activity of the VR2 polypeptide is in excess, several approaches are available. One approach comprises administering to a subject in need thereof an inhibitor compound (antagonist) as hereinabove described, optionally in combination with a pharmaceutically acceptable carrier, in an amount effective to inhibit the function of the VR2 polypeptide, such as, for example, by blocking the binding of ligands, substrates, receptors, enzymes, etc., or by inhibiting a second signal, and thereby alleviating the abnormal condition. In another approach, soluble forms of the VR2 polypeptide still capable of binding the ligand substrate, enzymes, receptors, etc., in competition with endogenous polypeptide may be administered. Typical examples of such competitors include fragments of the VR2 polypeptide.

In still another approach, expression of the gene encoding endogenous VR2 polypeptide can be inhibited using expression blocking techniques. Known such techniques involve the use of antisense sequences, either internally generated or externally administered (see, for example, O'Connor, J. Neurochem., (1991) 56:560 in Oligodeoxynucleotides as Antisense Inhibitors of Gene Expression, CRC Press, Boca Raton. Fla. (1988)). Such antisense polynucleotides are designed to comprise the antisense sequence of a polynucleotide encoding a VR2 polypeptide, or a fragment thereof. A VR2 encoding polynucleotide can include a DNA or an RNA, for example a mRNA.

Alternatively, oligonucleotides which form triple helices (“triplexes”) with the gene can be supplied (see, for example, Lee et al., Nucleic Acids Res., (1979) 6:3073; Cooney et al., Science (1988) 241:456; Dervan et al., Science (1991) 251:1360). These oligomers can be administered per se or the relevant oligomers can be expressed in vivo. Synthetic antisense or triplex oligonucleotides may comprise modified bases or modified backbones. Examples of the latter include methylphosphonate, phosphorothioate or peptide nucleic acid backbones. Such backbones are incorporated in the antisense or triplex oligonucleotide in order to provide protection from degradation by nucleases and are well known in the art. Antisense and triplex molecules synthesised with these or other modified backbones also form part of the present invention.

In addition, expression of the human VR2 polypeptide may be prevented by using ribozymes specific to the human VR2 mRNA sequence. Ribozymes are catalytically active RNAs that can be natural or synthetic (see for example Usman, N, et al. Curr. Opin. Struct. Biol., (1996) 6(4):527-33). Synthetic ribozymes can be designed to specifically cleave the human VR2 mRNAs at selected positions thereby preventing translation of the human VR2 mRNAs into functional polypeptide. Ribozymes may be synthesised with a natural ribose phosphate backbone and natural bases, as normally found in RNA molecules. Alternatively the ribozymes may be synthesised with non-natural backbones to provide protection from ribonuclease degradation, for example, 2′-O-methyl RNA, and may contain modified bases.

For treating abnormal conditions related to an under-expression of VR2 and its activity, several approaches are also available. One approach comprises administering to a subject a therapeutically effective amount of a compound which activates a VR2 polypeptide of the present invention, i.e. an agonist as described above, in combination with a pharmaceutically acceptable carrier, to thereby alleviate the abnormal condition. Alternatively, gene therapy may be employed to effect the endogenous production of VR2 by the relevant cells in the subject. For example, a polynucleotide of the invention may be engineered for expression in a replication defective retroviral vector, as discussed above. The retroviral expression construct may then be isolated and introduced into a packaging cell transduced with a retroviral plasmid vector containing RNA encoding a polypeptide of the present invention such that the packaging cell now produces infectious viral particles containing the gene of interest. These producer cells may be administered to a subject for engineering cells in vivo and expression of the polypeptide in vivo. For an overview of gene therapy, see Chapter 20, Gene Therapy and other Molecular Genetic-based Therapeutic Approaches, (and references cited therein) in Human Molecular Genetics, T Strachan and A P Read, BIOS Scientific Publishers Ltd (1996). Another approach is to administer a therapeutic amount of a VR2 polypeptide of the present invention in combination with a suitable pharmaceutical carrier.

In a further aspect, the present invention provides for pharmaceutical compositions comprising a therapeutically effective amount of a VR2 polypeptide, such as the soluble form of a VR2 polypeptide of the present invention, agonist/antagonist peptide, or small molecule compound, in combination with a pharmaceutically acceptable carrier or excipient. Such carriers include, but are not limited to, saline, buffered saline, dextrose, water, glycerol, ethanol, and combinations thereof. The invention further relates to pharmaceutical packs and kits comprising one or more containers filled with one or more of the ingredients of the aforementioned compositions of the invention. VR2 polypeptides and other compounds of the present invention may be employed alone or in conjunction with other compounds, such as therapeutic compounds.

The composition will be adapted to the route of administration, for instance by a systemic or an oral route. Preferred forms of systemic administration include injection, typically by intravenous injection. Other injection routes, such as subcutaneous, intramuscular, or intraperitoneal, can be used. Alternative means for systemic administration include transmucosal and transdermal administration using penetrants such as bile salts or fusidic acids or other detergents. In addition, if a VR2 polypeptide or other compounds of the present invention can be formulated in an enteric or an encapsulated formulation, oral administration may also be possible. Administration of these compounds may also be topical and/or localised, in the form of salves, pastes, gels, and the like.

It will be appreciated that the amount of a compound of formula (I) required for use in any treatment will vary not only with the particular compounds or composition selected but also with the route of administration, the nature of the condition being treated, and the age and condition of the patient, and will ultimately be at the discretion of the attendant physician. Suitable dosages, however, are in the range of 0.1 to 100 g/kg of subject. Wide variations in the needed dosage, however, are to be expected in view of the variety of compounds available and the differing efficiencies of various routes of administration. For example, oral administration would be expected to require higher dosages than administration by intravenous injection.

Variations in these dosage levels can be adjusted using standard empirical routines for optimisation, as is well understood in the art.

Polypeptides used in treatment can also be generated endogenously in the subject, in treatment modalities often referred to as “gene therapy” as described above. Thus, for example, cells from a subject may be engineered with a polynucleotide, such as a DNA or RNA, to encode a VR2 polypeptide ex vivo, and for example, by the use of a retroviral plasmid vector. The cells are then introduced into the subject.

The following definitions are provided to facilitate understanding of certain terms used frequently hereinbefore.

“Antibodies” as used herein includes polyclonal and monoclonal antibodies, chimeric, single chain, and humanised antibodies, as well as Fab fragments, including the products of an Fab or other immunoglobulin expression library.

“Isolated” means altered “by the hand of man” from the natural state. If an “isolated” composition or substance occurs in nature, it has been changed or removed from its original environment, or both. For example, a polynucleotide or a polypeptide naturally present in a living animal is not “isolated”, but the same polynucleotide or polypeptide separated from the coexisting materials of its natural state is “isolated”, as the term is employed herein.

“Polynucleotide” generally refers to any polyribonucleotide or polydeoxyribonucleotide, which may be unmodified RNA or DNA or modified RNA or DNA. “Polynucleotides” include, without limitation, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term “polynucleotide” also includes DNAs or RNAs containing one or more modified bases and DNAs or RNAs with backbones modified for stability or for other reasons. “Modified” bases include, for example, tritylated bases and unusual bases such as inosine. A variety of modifications may be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

“Polypeptide” refers to any peptide or protein comprising two or more amino acids joined to each other by peptide bonds or modified peptide bonds, i.e. peptide isosteres. “Polypeptide” refers to both short chains, commonly referred to as peptides, oligopeptides or oligomers, and to longer chains, generally referred to as proteins.

Polypeptides may contain amino acids other than the 20 gene-encoded amino acids. “Polypeptides” include amino acid sequences modified either by natural processes, such as post-translational processing, or by chemical modification techniques which are well known in the art. Such modifications are well-described in basic texts and in more detailed monographs, as well as in a voluminous research literature. Modifications may occur anywhere in a polypeptide, including the peptide backbone, the amino acid side-chains and the amino or carboxyl termini. It will be appreciated that the same type of modification may be present to the same or varying degrees at several sites in a given polypeptide. Also, a given polypeptide may contain many types of modifications. Polypeptides may be branched as a result of ubiquitination, and they may be cyclic, with or without branching. Cyclic, branched and branched cyclic polypeptides may result from post-translation natural processes or may be made by synthetic methods. Modifications include acetylation, acylation, ADP-ribosylation, amidation, biotinylation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination (see, for instance, Proteins-Structure and Molecular Properties, 2nd Ed., T. E. Creighton, W. H. Freeman and Company, New York, 1993; Wolds F., Post-translational Protein Modifications: Perspectives and Prospects, pgs. 1-12 in Post-translational Covalent Modification of Proteins, B. C., Johnson, Ed., Academic Press, New York, 1983; Seifter et al., “Analysis for protein modifications and nonprotein cofactors”, Meth. Enzymol, (1990) 182:626-646 and Rattan et al., “Protein Synthesis; Post-translational Modifications and Aging”. Ann. NY Acad Sci, (1992) 663:48-62).

“Variant” refers to a polynucleotide or polypeptide that differs from a reference polynucleotide or polypeptide, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, and deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques or by direct synthesis.

“Identity”, as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including but not limited to those described in Computational Molecular Biology, Lesk, A. M., ed., Oxford University Press, N.Y., 1988; Biocomputing: Informatics and Genome Projects, Smith, D. W., ed., Academic Press, N.Y., 1993; Computer Analysis of Sequence Data, Part I, Griffin, A. M., and Griffin, H. G., eds., Humana Press, N.J., 1994; Sequence Analysis in Molecular Biology, von Heinje, G., Academic Press, N.Y., 1987; and Sequence Analysis Primer, Gribskov, M. and Devereux, J., eds., M Stockton Press, N.Y., 1991; and Carillo, H., and Lipman, D., SIAM J. Applied Math., 48:1073 (1988). Preferred methods to determine identity are designed to give the largest match between the sequences tested. Methods to determine identity and similarity are codified in publicly available computer programs. Preferred computer program methods to determine identity and similarity between two sequences include, but are not limited to, the GCG program package (Devereux, J., et al., Nucleic Acids Res., 12(1):387 (1984)), BLASTP, BLASTN, and FASTA (Atschul, S. F. et al., J. Molec. Biol., 215:403-410 (1990). The BLAST X program is publicly available from NCBI and other sources (BLAST Manual, Altschul, S., et al., NCBI NLM NIH Bethesda, Md. 20894). The well-known Smith Waterman algorithm may also be used to determine identity.

By way of example, a polynucleotide sequence of the present invention may be identical to the reference sequence of SEQ ID NO: 1, that is be 100% identical, or it may include up to a certain integer number of nucleotide alterations as compared to the reference sequence. Such alterations are selected from the group consisting of at least one nucleotide deletion, substitution, including transition and transversion, or insertion, and wherein said alterations may occur at the 5′ or 3′ terminal positions of the reference nucleotide sequence or anywhere between those terminal positions, interspersed either individually among the nucleotides in the reference sequence or in one or more contiguous groups within the reference sequence. The number of nucleotide alterations is determined by multiplying the total number of nucleotides in SEQ ID NO: 1 by the numerical percent of the respective percent identity (divided by 100) and subtracting that product from said total number of nucleotides in SEQ ID NO: 1, or:
nₙ≧xₙ−(xₙ·y)
wherein nₙ is the number of nucleotide alterations, xₙ is the total number of nucleotides in SEQ ID NO: 1, and y is, for instance, 0.70 for 70%, 0.80 for 80%, 0.85 for 85%, 0.90 for 90%, 0.95 for 95%, etc., and wherein any non-integer product of xₙ and y is rounded down to the nearest integer prior to subtracting it from xₙ. Alterations of a polynucleotide sequence encoding the polypeptide of SEQ ID NO: 2 may create nonsense missense or frameshift mutations in this coding sequence and thereby alter the polypeptide encoded by the polynucleotide following such alterations.

Similarly, a polypeptide sequence of the present invention may be identical to the reference sequence of SEQ ID NO: 2, that is be 100% identical, or it may include up to a certain integer number of amino acid alterations as compared to the reference sequence such that the % identity is less than 100%. Such alterations are selected from the group consisting of at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in SEQ ID NO: 2 by the numerical percent of the respective percent identity (divided by 100) and then subtracting that product from said total number of amino acids in SEQ ID NO: 2, or:
nₐ≧xₐ−(xₐ·y)
wherein nₐ is the number of amino acid alterations, xₐ is the total number of amino acids in SEQ ID NO: 2, and y is, for instance 0.70 for 70%, 0.80 for 80%, 0.85 for 85%, etc., and wherein any non-integer product of xₐ and y is rounded down to the nearest integer prior to subtracting it from xₐ.

“Homolog” is a generic term used in the art to indicate a polynucleotide or polypeptide sequence possessing a high degree of sequence relatedness to a subject sequence. Such relatedness may be quantified by determining the degree of identity and/or similarity between the sequences being compared as hereinbefore described. Falling within this generic term are the terms “ortholog” , meaning a polynucleotide or polypeptide that is the functional equivalent of a polynucleotide or polypeptide in another species, and “paralog” meaning a functionally similar sequence when considered within the same species.

“Fusion protein” refers to a protein encoded by two, often unrelated, fused genes or fragments thereof. For instance, employing an immunoglobulin Fc region as a part of a fusion protein is advantageous for use in therapy and diagnosis resulting in, for example, improved pharmacokinetic properties. On the other hand, for some uses it would be desirable to be able to delete the Fc part after the fusion protein has been expressed, detected and purified.

Localisation of VR2 (TRPV2) in primate brain was performed as follows:

### MATERIALS AND METHODS

Antibodies

To raise specific polyclonal antisera, rabbits were immunized with a short synthetic peptide specific to the human VR2 C-terminal sequence conjugated to keyhole limpet haemocyanin. Resultant antisera were assessed using enzyme linked immunosorbent assay and dot-blot to evaluate extent and strength of the immune response made to the immunogen. Subsequently, sera were affinity-purified for use in immunohistochemical techniques. For single-, double- and triple-labelling investigations, previously characterized sera specific for vasopressin (VP) and oxytocin (OXY) were obtained from commercial sources.

Immunohistochemistry: Single-Labelling Using Colorimetric and Fluorescent Detection

10% formal-saline-fixed, paraffin-embedded primate brain sections were dewaxed in xylene, incubated in 0.3% hydrogen peroxide in methanol, rehydrated through an ethanol series and microwaved in citrate buffer, pH 6.0 in order to retrieve antigenicity. Sections were then blocked in 5% normal serum in phosphate buffered saline (PBS)-Triton X-100, prior to overnight incubation at +4° C. in affinity-purified antiserum in the presence or absence of 20-fold excess immunizing peptide (for VR2-specific antiserum characterization). Subsequently, sections were washed in PBS and immunoreactivity detected using biotinylated secondary antibodies, followed by Avidin Biotin Complex (ABC) and visualized using diaminobenzidine/hydrogen peroxide. All single-labelled colorimetric immunohistochemical detection steps were performed on a robotic immunostainer to increase intersection staining consistency.

For fluorescent detection, immunoreactivity was detected using fluorescein isothiocyanate (FITC) conjugated secondary reagents and visualized using confocal microscopy.

Confocal Microseopy: Double- and Triple-Labelling

Slides were processed as detailed above before incubation overnight with affinity-purified antisera. Visualization for double- and triple-labelling was carried out as follows: rabbit anti-VR2 primary antibodies were detected using biotinylated goat anti-rabbit secondary antibody, followed by a tertiary layer of Texas Red Streptavidin; guinea pig anti-VP primary antibodies were detected using anti-guinea pig FITC conjugated sera. Double- and triple-labelling studies involving the detection of oxytocin were carried out using both guinea pig- and mouse-anti-oxytocin antibodies. When detecting sera raised in guinea pig (e.g. for double-labeling), a FITC-conjugated anti-guinea pig secondary antibody was used, whilst when using mouse anti-oxytocin antibodies (e.g. for triple-labelling) anti-OXY primary antibodies were detected using a Cy5.5-conjugated anti-mouse secondary antiserum. Sections were mounted in Immu-Mount (Shandon, Pa., USA) and visualized using a Multi Band Confocal Imaging Spectrophotometer (Leica TCS SP, Wetzlar, Germany).

### RESULTS

Single Label Immunohistochemistry

Using single-labelling immunohistochemistry, VR2-like-immunoreactive immunoreactive material (-ir) was abundantly, yet discretely localized in primate brain. Highly intense VR2-ir was observed in paraventricular nucleus of the hypothalamus (PVN), supraoptic nucleus (SON) and suprachiasmatic nucleus (SCN) (see FIGS. 2 and 3). These expression data suggest that VR2 may have neuroendocrine regulatory function(s), as these regions are the neuroanatomical location of oxytocinergic and vasopressinergic neurons, as well as those that express corticotrophin releasing factor (CRF). This was subsequently confirmed using immunohistochemistry using sera specific for oxytocin (OXY) and vasopressin (VP) in sections from the same primates used to investigate VR2 expression.

Double- and Triple-Labelliing Confocal Microscopy

To gain further understanding of the extent of co-expression of VR2 with OXY and VP, single-, double- and triple-labelling confocal immunohistochemistry was carried out (see FIGS. 4 and 5). In brief, VR2-ir was shown by double-labelling to be almost entirely restricted to oxytocinergic and vasopressinergic neurons: in both PVN and SON; most, if not all, OXY-positive cells expressed VR2-ir, but not all VR2-ir cells were OXY-positive; likewise, most, if not all, VP-positive cells expressed VR2-ir, but not all VR2-ir cells were VP positive. These data further implicated the involvement of VR2 in hypophyseal function.

To conclusively determine exclusivity of expression of VR2 to OXY- and VP-expressing neurons, triple-labelling confocal immunofluourescence was carried out on PVN, SON, suprachiasmatic nucleus (SCN) and pituitary sections. The resultant data confirmed previous single- and double-labelling investigations. Briefly, cell counts across all 3 regions, PVN, SON and SCN were consistent; approximately 50% of all cells counted were triple labelled for VR2-/VP-/OXY-ir. Except for a very few number of cells, all VR2-ir positive cells were also labelled for either VP-ir and/or OXY-ir, i.e. there were little, if any, singlelabelled VR2-ir cells, no cells labelled for VP-ir alone, and no cells double labelled for VP-ir and OXY-ir alone. These data convincingly indicate involvement of VR2 in hypophyseal functions utilizing VP and OXY including, anxiety and depression, diuresis, erectile function, lactation, parturition and sleep, and related behaviours, including schizophrenia.

1-22. (canceled)

23. A method for the treatment of anxiety and/or depression and/or circadian rhythm disorders which comprises administration of an effective amount of a compound selected from: (a) a VR2 polypeptide; (b) a compound which modulates the activity of a VR2 polypeptide; (c) a polynucleotide encoding a VR2 polypeptide; or (d) an antisense polynucleotide to a polynucleotide encoding a VR2 polypeptide, to a patient in need of such treatment.

24. A method of claim 23 wherein said anxiety is a disorder selected from panic disorder with or without agoraphobia, agoraphobia without history of panic disorder, specific phobias, social phobias and obsessive-compulsive disorders.

25. A method of claim 23 wherein said anxiety is a disorder selected from anxiety disorders induced by alcohol, amphetamines, caffeine, cannabis, cocaine, hallucinogens, inhalants, phencyclidine, sedatives, hypnotics, anxiolytics and other substances, and adjustment disorders with anxiety.

26. A method of claim 23 wherein said depression is a disorder selected from single or recurrent major depressive episodes, with or without pyschotic features, catatonic features, melancholic features, atypical features or postpartum onset and, in the case of recurrent episodes, with or without interepisode recovery and with or without seasonal pattern.

27. A method of claim 23 wherein said depression is a disorder selected from dysthymic disorder with early or late onset and with or without atypical features; dementia of the Alzheimer's type, with early or late onset, with depressed mood; vascular dementia with depressed mood; mood disorders induced by alcohol, amphetamines, cocaine, hallucinogens, inhalants, opioids, phencyclidine, sedatives, hypnotics, anxiolytics and other substances; schizoaffective disorder of the depressed type; and adjustment disorder with depressed mood.

28. A method of claim 23 wherein said depression is the result of a general medical condition selected from myocardial infarction, diabetes, miscarriage and abortion.

29. A method of claim 23 for achieving a circadian rhythm phase-shifting effect in a mammal.

30. A method of claim 23 for resetting the internal circadian clock in a mammal.

31. A method of claim 23 for shortening the time of reintrainment of circadian rhythms in a mammal.

32. A method of claim 23 for enhancing or improving sleep I quality and/or preventing and/or treating sleep disorders and sleep disturbances in a mammal.!

33. A method of claim 23 for increasing sleep efficiency and augmenting sleep maintenance in a mammal.

34. A method of claim 23 for the prevention or treatment of a circadian rhythm disorder in a mammal, which disorder is selected from the group consisting of: time-zone change (jet-lag) syndrome, shift-work sleep disorder, delayed sleep-phase syndrome, advanced i sleep-phase syndrome, and non-24-hour sleep-wake disorder.

35. A method of claim 23 for the prevention or treatment of a circadian rhythm disorder in a mammal, which disorder is selected from the group consisting of Disorders of Initiating and Maintaining Sleep (insomnias) (“DIMS”), childhood onset DIMS, nocturnal myoclonus and restless legs and non specific REM disturbances as seen in ageing.

36. A method for the treatment of pre-term labour, erectile dysfunction, hypertension and/or eclampsia, and associated disorders or schizophrenia which comprises administration of an effective amount of a compound selected from: (a) a VR2 polypeptide; (b) a compound which modulates the activity of a VR2 polypeptide; (c) a polynucleotide encoding a VR2 polypeptide; or (d) an antisense polynucleotide to a polynucleotide encoding a VR2 polypeptide, to a patient in need of such treatment.

37. A method of claim 36 for the treatment of pre-term labour, erectile dysfunction, hypertension and/or eclampsia, and associated disorders.

38. A method of claim 36 inducing myometrial relaxation, preventing pre- term labour, stopping labour, and/or treating or preventing dysmenorrhea.

39. A method of claim 36 for the treatment or prevention of erectile dysfunction and/or impotence.

40. A method of claim 36 for the treatment or prevention of hypertension and/or congestive heart failure, inducing diuresis, and/or inhibiting platelet agglutination.

41. A method of claim 36 for the treatment or prevention of pre-eclampsia and/or eclampsia.

42. A method of claim 36 wherein the compound which modulates the activity of a VR2 polypeptide is an antagonist.

43. A method of claim 23 wherein the compound which modulates the activity of a VR2 polypeptide is an antagonist.

44. A method of claim 23 wherein the compound is a VR2 polypeptide which comprises a polypeptide having at least 95% identity to the VR2 polypeptide of SEQ ID NO: 2.

45. A method of claim 44 wherein the compound is the VR2 polypeptide of SEQ ID NO: 2.

46. A method of claim 23 wherein the compound comprises a polynucleotide encoding a polypeptide having at least 95% identity with the amino acid sequence of SEQ ID NO: 2.

47. A method of claim 46 wherein the polynucleotide 2 comprises a polynucleotide having at least 95% identity with the polynucleotide of SEQ ID NO: 1.

48. A method of claim 47 wherein the polynucleotide has the polynucleotide sequence of SEQ ID NO: 1.

49. A method of claim 36 wherein the compound which modulates the activity of a VR2 polypeptide is an antagonist.

50. A method of claim 36 wherein the compound is a VR2 polypeptide which comprises a polypeptide having at least 95% identity to the VR2 polypeptide of SEQ ID NO: 2.

51. A method of claim 50 wherein the compound is the VR2 polypeptide of SEQ ID NO: 2.

52. A method of claim 36 wherein the compound comprises a polynucleotide encoding a polypeptide having at least 95% identity with the amino acid sequence of SEQ ID NO: 2.

53. A method of claim 52 wherein the polynucleotide 2 comprises a polynucleotide having at least 95% identity with the polynucleotide of SEQ ID NO: 1.

54. A method of claim 53 wherein the polynucleotide has the polynucleotide sequence of SEQ ID NO: 1.
